(19) United States
(12) Patent Application Publication
(10) Pub. No.: US 2020/0155631 A1
(43) Pub. Date: May 21, 2020

(54) METHOD OF STABILIZING MIND AND BODY WITH DISTILLED EXTRACT OF MOUNTAIN-CULTIVATED PANAX GINSENG

(71) Applicants: Jayeonae Co., Ltd., Sancheong-gun (KR); Bioblend Co., Ltd., Hamyang-gun (KR)

(72) Inventors: Min Cheol Kang, Hamyang-gun (KR); Kye Man Cho, Jinju-si (KR); Mi Jung Jang, Sancheong-gun (KR)

(73) Assignees: Jayeonae Co., Ltd., Sancheong-gun (KR); Bioblend Co., Ltd., Hamyang-gun (KR)

(21) Appl. No.: 16/556,222

(22) Filed: Aug. 29, 2019

(30) Foreign Application Priority Data
Nov. 21, 2018 (KR) 10-2018-0144928

(51) Int. Cl. A61K 36/258; A61K 9/00; A61K 8/9789; A61P 25/22

(52) U.S. Cl. CPC A61K 36/258; A61K 9/0073; A61P 25/22; A61K 8/9789; A61K 9/0056

(57) ABSTRACT

Disclosed herein is a method of stabilizing mind and body using a distilled extract of mountain-cultivated Panax ginsengs. The distilled extract of mountain-cultivated Panax ginsengs, when it is inhaled into human being, relatively increases the theta and alpha waves in the brain waves and relatively decreases the beta and gamma waves, thereby inducing the effects of mental and physical stability and relaxation of human beings. The distilled extract of mountain-cultivated Panax ginsengs according to the present disclosure is useful as a fragrance material to impart the effects of mental and physical stability and relaxation for a perfume composition, a functional food, a cosmetic product, and a hygiene product.

### CROSS-REFERENCE TO RELATED APPLICATION(S)

This application claims priority from Korean Patent Application No. 10-2018-0144928, filed on Nov. 21, 2018, in the United States Patent and Trademark office, the disclosure of which is incorporated herein by reference in its entirety.

### TECHNICAL FIELD

The present disclosure relates to a method of stabilizing mind and body using a distilled extract of mountain-cultivated Panax ginseng, and more particularly, to a method for stabilizing body and mind and relieving tension, which comprises treating a distilled extract of mountain-cultivated Panax ginseng to a subject to relatively increase the theta and alpha waves and to relatively reduce the beta and gamma waves in the subject brain.

### RELATED ART

Electroencephalography (EEG) is an electrophysiological monitoring method to record electrical activity of the brain. EEG measures voltage fluctuations resulting from ionic current within the neurons of the brain. EEG refers to the recording of the brain's spontaneous electrical activity over a period of time, as recorded from multiple electrodes placed on the scalp. EEG reflects varying activity of the brain. EEG expresses spontaneous potential fluctuations of the cerebral cortex in terms of time and space, and it is widely used in neuroscience research because of its excellent temporal resolution. EEG appears from several μV to 75 μV in awake person. EEG signal is mostly caused by differential post-synaptic potentials occurring in the cell bodies and large dendrites (Korean Patent Application Publication No. 10-2015-0030513).

Brain waves are categorized as theta waves, alpha waves, beta waves, and gamma waves according to the waveforms. Theta wave is a waveform that can be detected mainly when sleepy, not in deep sleep. Theta wave can also be detected when being engaged in entertaining jobs or when performing artistic work. Alpha wave is a waveform that is observed when relaxing or when closing the eyes while awake. Alpha wave is also observed when being in a mental state of stability, when being comfortable and relaxed, when listening to quiet meditative music, when being in meditation, or when being habituated to external stimuli. Beta wave is a waveform that can be detected when the brain nerves consume a lot of energy. Beta waves can also be detected when being engaged in learning activities that actively take information from outside in a state of awakening. Gamma wave is a waveform that is observed when mental power is concentrated by activating the nervous resources of transcendental state or new state of consciousness. Gamma wave is activated at a high level of cognitive function such as reasoning or judgment, and is also detected when emotionally more irritable and anxious.

The volatile flavor compounds contained in plant extracts pass through the nasal passages to reach the olfactory epithelium, and the olfactory nerve cells in the olfactory epithelium sense them and transmit electrical signals to the brain. It is known that unique brain waves are generated in a human brain for each plant extract. Therefore, there is a demand for new plant extracts containing volatile flavor compounds capable of improving brain waves, which are highly utilizable in a variety of fields such as perfumery, functional foods, cosmetics, and hygiene products.

Panax Ginseng is classified into Panax ginseng, mountain Panax ginseng, and mountain-cultivated Panax ginseng according to growth or cultivation environment and morphology. In particular, the mountain-cultivated Panax ginseng refers to a Panax ginseng (including dried one) that is cultivated without the establishment of artificial facilities such as shading films in the mountain areas defined in Article 2, Paragraph 1 of the Korean Mountain Management Act. The mountain-cultivated Panax ginseng is generally cultivated by using seeds or sprouts of wild ginseng in the mountains. Especially, the mountain-cultivated Panax ginseng is a forest product that is cultivated in the forests in a natural state and for a long period of time, in a clean (organic, without pesticide) form. Therefore, the mountain-cultivated panax ginseng is cultivated and managed so as to ensure naturalness and cleanliness in the Korean Forest Promotion Act. In the case of Panax ginsengs, most of them have from 3 to 7 outcrops (head part), but the mountain-cultivated Panax ginseng has more outcrops depending on its age. The roots of Panax ginseng are thick and short, but the mountain-cultivated Panax ginseng has long and thin roots. It is known that the mountain-cultivated Panax ginseng has ginsenosides with the higher contents than Panax ginseng.

To date, studies on a mountain-cultivated Panax ginseng have focused on ginsenosides, polyphenol compounds and amino acid-based substances among non-ginsenoside compounds, which have important physiological activities. However, using method of volatile flavor components (aroma-active compounds) derived from the mountain-cultivated Panax ginseng has not yet developed.

### SUMMARY

An object of the present disclosure is to provide a method of relatively increasing the theta and alpha waves of a subject and relatively reducing the beta and gamma waves of the subject to stabilize its mind and body and relieve tension, which comprises a step of treating a distilled extract of mountain-cultivated Panax ginseng.

Another object of the present disclosure is to provide a method for producing a distilled extract of mountain-cultivated Panax ginseng.

Still another object of the present disclosure is to provide uses of a distilled extract of mountain-cultivated Panax ginseng in the industry fields of perfumery, functional foods, cosmetics, and hygiene products.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a process for producing a distilled extract of mountain-cultivated Panax ginseng according to an embodiment of the present disclosure.

FIG. 2 is a GC chromatogram of volatile flavor components of the distilled extract of mountain-cultivated Panax ginseng according to the present disclosure.

FIG. 3A is an EEG map showing the change in the theta waves of the subject before and after inhalation of the distilled extract of mountain-cultivated Panax ginseng according to the present disclosure. Value of relative power were expressed by color in high-to-low order: red, orange, yellow, green, blue, and violet.

FIG. 3B is an EEG map showing the change in the alpha waves of the subject before and after inhalation of the distilled extract of mountain-cultivated Panax ginseng according to the present disclosure. Value of relative power were expressed by color in high-to-low order: red, orange, yellow, green, blue, and violet.

FIG. 3C is an EEG map showing the change in the beta waves of the subject before and after inhalation of the distilled extract of mountain-cultivated Panax ginseng according to the present disclosure. Value of relative power were expressed by color in high-to-low order: red, orange, yellow, green, blue, and violet.

FIG. 3D is an EEG map showing the change in the gamma waves of the subject before and after inhalation of the distilled extract of mountain-cultivated Panax ginseng according to the present disclosure. Value of relative power were expressed by color in high-to-low order: red, orange, yellow, green, blue, and violet.

### DETAILED DESCRIPTION

In order to accomplish the above object, the present disclosure provides a preparing method of a distilled extract of mountain-cultivated Panax ginseng.

In the present disclosure, the distilled extract of mountain-cultivated Panax ginseng can be prepared by a extractive distillation method comprising the following steps:

i) adding 10 to 30 times (v/w) of water to mountain-cultivated Panax ginsengs and heating the resulting mixture at the temperature of 60 to 80° C. for 15 to 45 minutes, and then performing extractive distillation at 100° C. to take a distillate (distilled liquid) in the amount of 30 to 40 wt % of the mixture; and

ii) adjusting salinity and pH of the distillate.

Step i) Extractive Distillation

In the present disclosure, it is preferable to use mountain-cultivated Panax ginsengs which are cultivated for at least three years at a field of an altitude of 500 m or higher.

In the present disclosure, the mountain-cultivated Panax ginsengs are washed three times in flowing water, and then washed twice more in the third-order distilled water. The washed mountain-cultivated Panax ginsengs are transferred to a wicker to remove water. The use of the third-order distilled water for washing makes the inorganic substances on the surface of the mountain-cultivated ginsengs completely removed, so that the extractive distillation can be more efficiently performed.

After removing heads of the washed mountain-cultivated Panax ginsengs, they are cut out in the size of 0.5 to 1 cm for efficient extractive distillation. The heads are added every year as the ginsengs grow, in which are accumulated a large amount of calcium oxalate. The calcium oxalate produces vomiting substances when it comes in contact with gastric juice. Accordingly, it is preferable to remove the heads from the washed mountain-cultivated Panax ginsengs.

It is preferable to add distilled water to mountain-cultivated Panax ginsengs in the amount of 10 to 30 times (v/w). When the distilled water is added at less than 10 times, the extractive distillation may not be sufficient. When the distilled water is added more than 30 times, it is economically disadvantageous because it takes a long time to extract and obtain effective components.

The mixture of distilled water and mountain-cultivated Panax ginsengs is preferably heated at 60 to 80° C. for 15 to 45 minutes. If the heating temperature and time is respectively lower than 60° C. and less than 15 minutes, volatile flavor components cannot be extracted sufficiently. If the heating temperature and time exceed respectively 80° C. and 45 minutes, components having toxicity can be generated from a low molecular chemical reaction.

Extractive distillation is carried out by heating the mixture at 100° C. to collect the distillate to the amount of 30-40% of the total weight of the mixture.

When the collection rate of the distillate is less than 30%, it is necessary to dilute the distillate because the volatile flavor components are excessively concentrated. If the collection rate is more than 40%, the concentration of the volatile flavor components may not be low and the concentration process may be necessary.

The length of the cooling tower of a distiller is preferably about 45 to 75 cm, and the cooling temperature is preferably kept at −10 to 0° C. When the length of the cooling tower is less than 45 cm and more than 75 cm, the efficiency of collection of volatile flavor components is lowered.

Step ii) Salinity and pH Adjustment

The salinity and pH of the distillate from the above step are adjusted to get a distilled extract of mountain-cultivated Panax ginsengs of the present disclosure.

The adjustment of salinity are performed by adding 0.5˜1.0 wt % of salt (NaCl) to the distillate and mixing well with a stirrer. Then pH of the distillate is adjusted to 7.0 to 7.2 by adding an acid among citric acid, malic acid and tartaric acid.

When salt is added in an amount of less than 0.5% by weight, the extracted components may be unstable. When salt is added in an amount exceeding 1.0% by weight, the concentration of the acid added during the pH adjustment may become excessive and the extracted components may become unstable.

To the distilled extract, a vitamin complex, an amino acid agent, a sweetener, a flavoring agent and/or a preservative may be further added, if necessary.

In the present disclosure, the distilled extract of mountain-cultivated Panax ginsengs contains β-Elemene, β-Panasinsene, Calarene, Caryophyllene, 2,3,5,6-tetramethylpyrazine, 4-1-methylethyl benzaldehyde, and α-Gurjunene. Particularly, the distillate comprise 13% or more of β-Elemene, 17% or more of β-Panasinsene, 13% or more of Calarene, and 12% or more of Caryophyllene in the total volatile flavor components. The distilled extract according to the present disclosure is characterized by containing 2,3,5,6-tetramethylpyrazine, 4-1-methylethylbenzaldehyde, and α-Gurjunene, which have strong fragrance strength (Table 1).

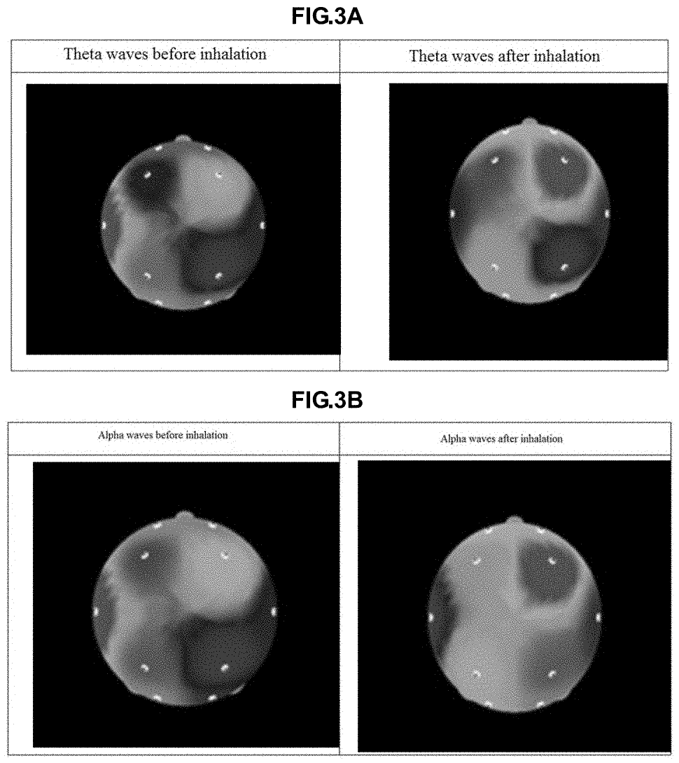

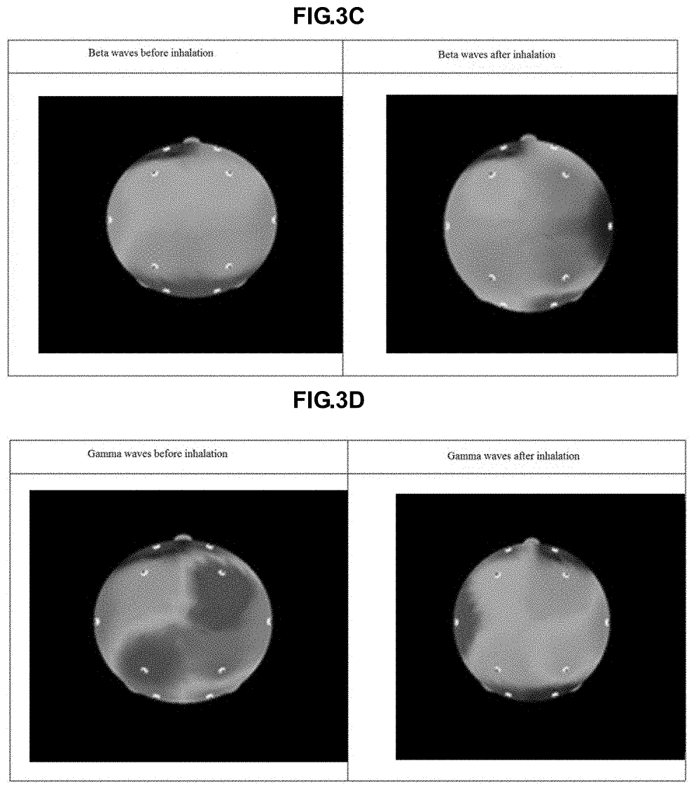

The distilled extract according to the present disclosure contains the above-mentioned volatile flavor components to relatively increase the theta and alpha waves of a subject, and relatively reduce the beta and gamma waves of the subject, resulting in the effects of mental and physical stability and relaxation (Test Example 2, FIG. 3).

According to yet another object of the present disclosure, the present disclosure provides a method for stabilizing body and mind and relieving tension, comprising the step of treating a distilled extract of mountain-cultivated Panax ginseng to a subject to relatively increase the theta and alpha waves and to relatively reduce the beta and gamma waves.

In the present disclosure, the subject is a mammal including a human, preferably a human.

In the present disclosure, the treatment comprises inhalation administration, topical administration or oral administration, preferably by inhalation administration.

In the present disclosure, the distilled extract of mountain-cultivated Panax ginsengs can be included in the perfumery fragrance.

In the present disclosure, the fragrance may further comprise one or more kinds of perfume ingredients that can be commonly used.

In the present disclosure, the distilled extract of mountain-cultivated Panax ginsengs can be comprised in food.

In the present disclosure, the food may be in the form of tea, beverage, jelly, concentrate or the like.

In the present disclosure, the distilled extract of mountain-cultivated Panax ginsengs can be comprised in cosmetics.

In the present disclosure, the cosmetics may be in the type of lotion, essence, cream, cleansing foam, cleansing water, pack, gel, powder, makeup base, body lotion, body cream, body oil, body essence, ointment, gel, cream, patch or spray.

In the present disclosure, the distilled extract of mountain-cultivated Panax ginsengs may be included in hygiene products.

In the present invention, the hygiene products may be shampoo, rinse, cologne, hair tonic, hair cream, indoor fragrance, soap, toothpaste, mouth wash, or toilet paper.

Effects

The distilled extract of mountain-cultivated Pananx ginsengs according to the present disclosure, when it is inhaled into a subject, relatively increases the theta and alpha waves in the brain waves and relatively decreases the beta and gamma waves, thereby inducing the effects of mental and physical stability and relaxation.

The distilled extract of mountain-cultivated Panax ginsengs according to the present disclosure is useful as a fragrance material to impart the effects of mental and physical stability and relaxation for a perfume composition, a functional food, a cosmetic product, and a hygiene product.

### EXAMPLES

Preparation Example 1. Preparation of distilled extract of mountain-cultivated Panax ginsengs

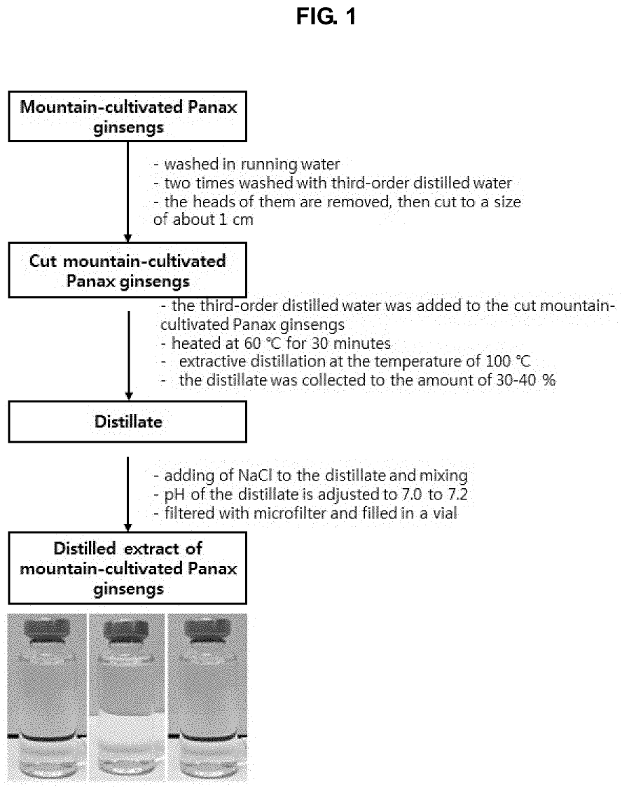

40 g of mountain-cultivated Panax ginsengs grown over three years were washed three times in running water, and two times in secondary washing with third-order distilled water. The washed mountain-cultivated Panax ginsengs were dried to remove water, and the heads of them were removed, and then cut to a size of about 1 cm. 800 ml of the third-order distilled water was added to the cut mountain-cultivated Panax ginsengs and the resulting mixture was heated at 60° C. for 30 minutes. Then the mixture was subjected to extractive distillation at the temperature of 100° C. in a distiller to collect the distillate (distilled liquid) through the cooling tower of the distiller up to 270 ml. 2.4 g of sodium chloride (NaCl) was added to the distillate. After mixing well with a stirrer, pH of the distillate was adjusted to 7.0˜7.2 by adding tartaric acid. The resulting distilled extract of mountain-cultivated Panax ginsengs was filtered with 0.02 μm filter and filled to a vial (FIG. 1).

### Test Example 1. Volatile Flavor Component Analysis and Olfactory Test

The volatile flavor components of the distilled extract of mountain-cultivated Panax ginsengs prepared in Preparation Example 1 were analyzed using a gas chromatograph mass spectrometer (GC-MS).

<Fragrance Component Adsorption>

SPME (solid-phase microextraction) was used for adsorption of the volatile flavor components. Headspace analysis method using SPME fibers (Supelco, Bellefonte, Pa., USA) coated with 100 μm polydimethylsiloxane (PDMS) was used for capturing the volatile flavor components of the distilled extract of mountain-cultivated Panax ginsengs. After 5 g of the sample (the distilled extract of mountain-cultivated Panax ginsengs) was placed in a fragrance bottle and sealed with an aluminum cap, the SPME fibers were injected into the bottle, and the fibers were exposed to the sample's vapor heated at 50° C. for 30 minutes for adsorption of the volatile flavor components.

<Volatile Flavor Component Analysis>

The SPME fibers adsorbed to the volatile flavor components were injected into a gas chromatograph equipped with a mass detector for the analysis of individual volatile flavor components. The analysis of the volatile flavor components using GC/MSD was performed using HP-5MS column (30 mm×0.25 mm i.d.×0.25 μm film thickness). The oven temperature program was allowed to stand at 40° C. for 5 minutes and then increased to 200° C. at a rate of 5° C. per minute. The injector temperature was 220° C. The volatile flavor components absolved to SPME were isolated. The flow rate of carrier gas helium was 1.0 ml/min, and the injection mode was splitless mode. Each component isolated from the total ionization chromatogram (TIC) of the sample was identified using NIST version 12, a library of volatile flavor components provided in the mass spectrum. Relative content of each volatile flavor component of the sample was identified based on the concentration and peak area of pentadcane used as a standard.

<Olfactory Test>

Each volatile flavor component separated by GC-MS for olfactory test was analyzed using an olfactory detection port with a heated mixing chamber (ODP 3, Gerstel, Linthicum, Md., USA) mounted on a GC-MSD for sniffing test of volunteers. Considering the difference of sense degree of smell felt among individuals and the decrease of sensitivity of smell with time, three skilled experimenters participated in the test and examined the intensity of each volatile flavor component.

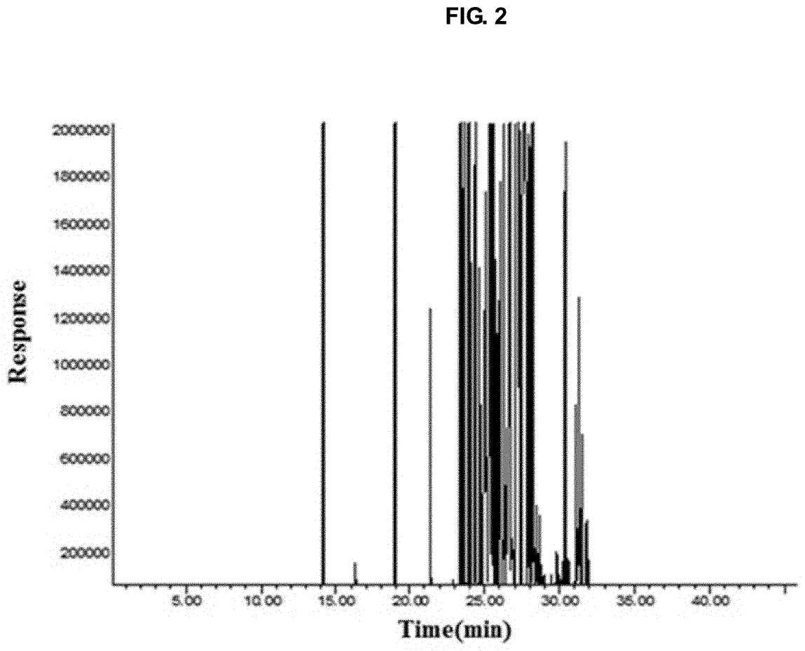

The test results are shown in Table 1 and FIG. 2.

TABLE 1

| | R.T. | | Content | Intensity |
|---|---|---|---|---|
| compound | (min) | R.I. | (%) | (O.I). |
| α-Pinene | 12.03 | 931 | 0.15 | |
| β-Myrcene | 13.98 | 988 | 0.03 | |
| 1-Piperidinecarboxaldehyde | 14.88 | 1015 | 0.02 | |
| Limonene | 15.20 | 1026 | 0.04 | |
| 2-Methoxy-3-1-methyl ethylpyrazine | 17.82 | 1109 | 0.02 | |
| 2,3,5,6 Tetramethyl pyrazine | 18.76 | 1142 | 0.02 | 1 |
| Bornyl acetate | 23.22 | 1298 | 0.03 | |
| Bicycloelemene | 23.86 | 1323 | 0.11 | |
| 1-Methyl ethyl benzene | 23.97 | 1328 | 3.97 | |
| Selina-4,11-diene | 24.46 | 1347 | 9.35 | |
| Drima-8,12-9,11-diene | 25.09 | 1371 | 3.42 | |
| β-Elemene | 25.65 | 1392 | 13.60 | 1 |
| 4-1-Methyl ethyl benzaldehyde | 25.79 | 1397 | 1.90 | 2 |
| α-Gurjunene | 26.07 | 1409 | 1.99 | |
| Calarene | 26.67 | 1434 | 12.94 | |
| β-Panasinsene | 27.27 | 1458 | 17.28 | |
| Alloaromadendrene | 27.38 | 1462 | 1.64 | |
| Caryophyllene | 27.65 | 1473 | 12.90 | |
| β-Selinene | 27.70 | 1475 | 3.77 | |
| β-Neodovene | 27.75 | 1477 | 3.17 | |
| Germacrene | 27.84 | 1481 | 0.22 | |
| Bicyclogermacrene | 28.28 | 1498 | 11.97 | |
| Butylatedhydroxytoluene | 28.50 | 1508 | 0.50 | |
| α-Amorphene | 28.56 | 1510 | 0.09 | |
| δ-Cadinene | 28.73 | 1517 | 0.39 | |
| 1,2,4,4-Tetramethyl cyclopentene | 28.85 | 1523 | 0.13 | |
| Cubedol | 28.98 | 1528 | 0.13 | |
| Nerolidol | 29.83 | 1565 | 0.21 | |

* R.T.: Retention time;
R.I.: Retention index;
O.I.: Odor Intensity

As shown in Table 1 and FIG. 2, 28 volatile flavor components were identified in the distilled extract of mountain-cultivated Panax ginsengs of the present disclosure. The distilled extract contained β-Panasinsene, β-Elemene, Calarene and at a high content of 17.28%, 13.60%, 12.94%, and 12.90%, respectively.

As a result of analysis of volatile flavor components through olfactory test, 2,3,5,6-tetramethyl pyrazine, 4-1-methylethyl benzaldehyde, and α-Gurjunene showed high intensity. In addition, 2,3,5,6-tetramethylpyrazine showed a sweet flavor, β-Elemene showed a herb flavor and 4-1-methylethyl benzaldehyde showed a sweet flavor.

### Test Example 2. Brain Indicator Analysis

<Selection of Subjects>

Twelve adults (male and female) were tested for differences in EEG before and after inhalation of the distilled extract of mountain-cultivated Panax ginsengs of the present disclosure. Before testing, the subjects were confirmed that they had not any history of mental illness, nor any experience of continuous medication and nasal surgery. Prior to the test, stimulant meals, smoking and drinking were prohibited to the subjects.

The test was carried out in the olfactory laboratory. The olfactory laboratory is equipped with intake and exhaust facilities for the purpose of reducing the olfactory adaptation, and installed with soundproofing devices to eliminate the influence of the external environment during the test. The internal temperature, humidity, and illuminance were kept constant during the test. At the end of each test on one condition, the laboratory was ventilated and the subjects were rested enough to avoid compliance with each volatile flavor component and fatigue.

<Brain Index Analysis>

Electrodes for EEG were attached to the prefrontal, frontal, temporal, parietal, and occipital lobes of the subjects using a 10-20 international standard electrode attachment method.

The distilled extract of mountain-cultivated Panax ginsengs of the present disclosure was provided in a 10-channel system at a distance of 1 to 2 cm below their nose, and their brain waves were measured before inhalation and while inhaling for 3 minutes. The collected data was summarized through processing program, and each EEG change was mapped through 3D EEG mapping program, and the results are shown in FIGS. 3A to 3D.

As shown in FIG. 3A, the subjects' theta waves increased in the temporal, parietal, and occipital lobes following the inhalation of the distilled extract of mountain-cultivated Panax ginsengs of the present disclosure. Theta waves appear when being sleepy or deeply meditated, suggesting that the subjects have become somewhat asleep.

As shown in FIG. 3B, the subject's alpha waves increased, especially in the right frontal lobe. In the case of alpha waves, they appear mainly when the brain function is relaxed (stable), suggesting that the subjects are emotionally stable.

As shown in FIG. 3C, the subjects' beta waves decreased in the temporal, parietal, and occipital lobes. Beta waves appear when mental activity is active. They are relatively reduced compared to other waves, suggesting that the subjects have relaxed tension and arousal.

As shown in FIG. 3D, the gamma wave of the subjects also decreased. Gamma-waves are mainly observed during intensive work. They are relatively reduced, suggesting that the subjects are relaxed.

From the above results, it can be seen that the distilled extract of mountain-cultivated Panax ginsengs of the present disclosure relatively increases the levels of theta and alpha waves in the brain waves and relatively reduces the beta and gamma waves to induce the effects of mental and physical stability and relaxation.

The examples disclosed in the present disclosure and the accompanying drawings are used not to limit but to describe the spirit of the present disclosure. The scope of the present disclosure is not limited only to the examples and the accompanying drawings. The scope of the present disclosure should be interpreted by the following claims and it should be interpreted that all spirits equivalent to the following claims fall within the scope of the present disclosure.

What is claimed is:

1. A preparation method of a distilled extract of mountain-cultivated Panax ginsengs for mental and physical stability and relaxation, comprising the following steps:
i) adding 10 to 30 times (v/w) of water to mountain-cultivated Panax ginsengs and heating the resulting mixture at the temperature of 60 to 80° C. for 15 to 45 minutes, and then performing extractive distillation at 100° C. to take the distillate in the amount of 30 to 40 wt % of the mixture; and
ii) adjusting salinity and pH of the distillate.

2. The preparation method of a distilled extract of mountain-cultivated Panax ginsengs of claim 1, wherein the salinity adjustment comprises adding 0.5-1.0 wt % of salt (NaCl) to the distillate.

3. The preparation method of a distilled extract of mountain-cultivated Panax ginsengs of claim 1, wherein the pH adjustment is carried out by adding at least one acid selected from the group consisting of citric acid, malic acid and tartaric acid to the distilled liquid to adjust the pH to 7.0 to 7.2.

4. A method for stabilizing body and mind and relieving tension, comprising a step of treating the distilled extract of mountain-cultivated Panax ginseng prepared by the method of claim 1 to a subject to relatively increase the theta and alpha waves and to relatively reduce beta and gamma waves.

5. The method for stabilizing body and mind and relieving tension of claim 4, wherein the treatment is conducted by inhalation administration.

6. The method for stabilizing body and mind and relieving tension of claim 4, wherein the distilled extract of mountain-cultivated Panax ginsengs is included in the perfumery fragrance.

7. The method for stabilizing body and mind and relieving tension of claim 4, wherein the distilled extract of mountain-cultivated Pananx ginsengs is included in food.

8. The method for stabilizing body and mind and relieving tension of claim 4, wherein the distilled extract of mountain-cultivated Panax ginsengs is included in cosmetics.

9. The method for stabilizing body and mind and relieving tension of claim 4, wherein the distilled extract of mountain-cultivated Panax ginsengs is hygiene products.

10. The method for stabilizing body and mind and relieving tension of claim 4, the subject is mammal.

11. The method for stabilizing body and mind and relieving tension of claim 4, the subject is human being.
